(19) United States
(12) Patent Application Publication
(10) Pub. No.: US 2023/0413887 A1
(43) Pub. Date: Dec. 28, 2023

(54) 2'-FUCOSYLLACTOSE FOR USE IN STIMULATING F. PRAUSNITZII ABUNDANCE

(71) Applicant: FrieslandCampina Nederland B.V., Amersfoort (NL)

(72) Inventor: Arjen Nauta, Wageningen (NL)

(21) Appl. No.: 18/039,075

(22) PCT Filed: Nov. 30, 2021
(86) PCT No.: PCT/EP2021/083489

§ 371 Date: May 26, 2023

(30) Foreign Application Priority Data
Dec. 1, 2020 (EP) 20211089.6
Mar. 5, 2021 (EP) 21161030.8
Apr. 30, 2021 (EP) 21171500.8

(51) Int. Cl. A23L 33/21; A23L 33/125

(52) U.S. Cl. CPC A23L 33/21; A23L 33/125

(57) ABSTRACT

Preparation of dietary fibers for increasing the abundance of Faecalibacteriumprausnitzii in the gastrointestinal tract of a human subject, the dietary fibers in said preparation consisting essentially of 2′-fucosyllactose (2′-FL) and optionally one or more further oligosaccharides selected from the group consisting of galacto-oligosaccharides, fructo-oligosaccharides, resistant starch, polydextrose, and human milk oligosaccharides other than 2′-fucosyllactose.

The present invention relates to a preparation of dietary fibers comprising at least 2′-fucosyllactose (2′-FL) for use in stimulating the abundance of Faecalibacterium prausnitzii in the gastrointestinal tract of human subjects.

The food we consume dictates to a large extent the nature of the bacterial species in our gut microbiota. Various beneficial gut bacteria consume dietary fibers such a oligo- and polysaccharides.

Human breast milk, for example, contains various oligosaccharides—classified as Human Milk Oligosaccharides (HMOs)—which have been shown to have beneficial impacts on infant health, more in particular in facilitating bacterial colonization in the gut and protection from pathogens. Although the amount and diversity of HMOs in human breast milk varies among woman, e.g. depending on geographic origin and genetic background, it can be said that human breast milk contains three major HMO types: fucosylated HMOs (35-50%), sialylated HMOs (12-14%), and nonfucosylated neutral HMOs (42-55%). Fucosylated HMOs include 2′-fucosyllactose (2′-FL), which is the most abundant HMO in human breast milk.

One of the gut microbiota species associated with health benefits is Faecalibacterium prausnitzii. As disclosed by M. Lebas, et al., Microorganisms 2020, 8, 1528, F. prausnitzii is a gram-negative bacterium that is prevalent and abundant in healthy subjects. Various studies have shown its effect on inflammatory bowel conditions such as Crohn's disease and ulcerative colitis.

K. Ganesan et al., Int. J. Mol. Sci., 2018, 19, 3720 reviewed the effect of F. prausnitzii on diabetes.

T. Tochio et al., Foods 2018, 7, 140 linked the abundance of F. prausnitzii with an improvement of symptoms associated with allergies such as atopic dermatitis.

The effect of F. prausnitzii on depression and anxiety was studied and reported by Z. Hao et al., Psychoneuroendocrinology, 2019; 104, 132-142.

Furthermore, F. prausnitzii seems to be a suitable biomarker for various gut conditions; see M. Lopez-Silas et al., The ISME Journal, 2017, 11, 841-852.

As shown by N. Salazar, Nutrients, 2019, 11, 1765, the abundance of F. prausnitzii decreases with age, especially above the age of 65.

The COVID-19 pandemic, also known as the coronavirus pandemic, is an ongoing pandemic of coronavirus disease 2019 (COVID-19) caused by severe acute respiratory syndrome coronavirus 2 (SARS-CoV-2). It was first identified in December 2019 in Wuhan, China. The World Health Organization declared the outbreak a Public Health Emergency of International Concern in January 2020 and a pandemic in March 2020. As of 4 Mar. 2021, more than 115 million cases have been confirmed, with more than 2.55 million deaths attributed to COVID-19, making it one of the deadliest pandemics in history.

Yeoh Y K, et al. (Gut 2021; 0:1-9. doi:10.1136/gutjnl-2020-323020) showed that gut microbiome composition was significantly altered in patients with COVID-19 compared with non-COVID-19 individuals irrespective of whether patients had received medication (p<0.01). Several gut commensals with known immunomodulatory potential such as Faecalibacterium prausnitzii were underrepresented in patients and remained low in samples collected up to 30 days after disease resolution. Moreover, this perturbed composition exhibited stratification with disease severity concordant with elevated concentrations of inflammatory cytokines and blood markers such as C reactive protein, lactate dehydrogenase, aspartate aminotransferase and gamma-glutamyl transferase. Associations between gut microbiota composition, levels of cytokines and inflammatory markers in patients with COVID-19 suggest that the gut microbiome is involved in the magnitude of COVID-19 severity, possibly via modulating host immune responses. Furthermore, the gut microbiota dysbiosis after disease resolution could contribute to persistent symptoms.

Hence, a treatment which can increase the abundance of F. prausnitzii in the gastrointestinal tract of a human subject is expected to have the effect of preventing, treating, or relieving symptoms of a COVID-19 or a SARS-CoV-2 infection.

The term “treatment”, in relation to a given disease or disorder, includes, but is not limited to, inhibiting the disease or disorder (e.g. arresting the development of the disease or disorder), relieving the disease or disorder (e.g. causing regression of the disease or disorder); and/or relieving a condition caused by or resulting from the disease or disorder (e.g. relieving, preventing or treating symptoms of the disease or disorder).

The term “prevention” in relation to a given disease or disorder means preventing the onset of disease development if none has yet occurred, preventing the disease or disorder from occurring in a subject that may be predisposed to the disorder or disease but has not yet been diagnosed as having the disorder or disease, and/or preventing further disease/disorder development if already present.

It is therefore an object of the present invention to provide a preparation and a nutritional composition that can increase the abundance of F. prausnitzii in the gastrointestinal tract of human subjects, preferably non-infants, more preferably adults, such as elderly in the age above 65.

A non-infant human subject in this document is defined as a human subject with an age of at least 3 years.

It is a further object of the present invention to provide a preparation and a nutritional composition for use in increasing the abundance of F. prausnitzii in the gastrointestinal tract of human subjects, preferably non-infants, more preferably adults, even more preferably elderly in the age above 65, suffering from or having an increased risk of suffering from COVID-19/a SARS-CoV-2 infection, inflammatory bowel conditions, diabetes, anxiety, and/or allergies such as atopic dermatitis.

It is another object of the invention to provide a preparation and a nutritional composition for use in reducing the severity of Covid-19 or a SARS-Cov-2 infection in human subjects, preferably non-infants, more preferably adults, most preferably elderly in the age above 65.

It has now been found that this object can be met by administering to these subjects a preparation of dietary fibers, the dietary fibers in said preparation consisting essentially of 2′-FL and optionally one or more further oligosaccharides selected from the group consisting of galacto-oligosaccharides, fructo-oligosaccharides, polydextrose, resistant starch, and human milk oligosaccharides other than 2′-fucosyllactose as the dietary fibers.

The term “consisting essentially of” as used in “dietary fibers in said preparation consisting essentially of 2′-fucosyllactose (2′-FL) and optionally one or more further oligosaccharides selected from the group consisting of galacto-oligosaccharides, fructo-oligosaccharides, resistant starch, polydextrose, and human milk oligosaccharides other than 2′-fucosyllactose” means that the preparation does not contain any material amount of dietary fibers other than the listed ones.

A “material amount” is defined as an amount not affecting the bacterial colonization in the gut and will in practice be <5 wt %, more preferably <1 wt %, based on dry weight of the dietary fibers. The preparation therefore does not contain any (material amount of) isomaltooligosaccharide; digestion resistant glucose oligomers with α-D-(1,6)-linkages.

In a preferred embodiment, the preparation consists of 2′-FL and the optional further oligosaccharides, as the only dietary fibers.

The preparation may contain compounds that cannot be classified as dietary fibers, such as water, mono-saccharides (e.g. fructose, glucose, galactose), sucrose, and/or lactose.

An in vitro study with fecal material of adult and elderly subjects has now shown that 2′-FL is able to increase the abundance of F. prausnitzii in such human subjects.

This is especially surprising considering the fact that the in vitro study performed by Cheng et al. (Frontiers in Microbiology, October 2020, Volume 11, article 569700) concluded that 2′-FL does not affect F. prausnitzii abundance in monocultures. Also in a co-culture with Bifidobacterium longum subsp. infantis, 2′-FL was not able to increase the abundance of F. prausnitzii. Apparently and surprisingly, following the present in vitro study, in co-cultures representative of the gut microbiome 2′-FL is able to increase the abundance of F. prausnitzii.

Furthermore, a study in rats showed a decrease in F. prausnitzii abundance as a result of 2′-FL administration (F. Cheilat et al., Nutrients 2020, 12, 1532).

And although US 2018/0015032 reported an enhanced level of F. prausnitzii in adults humans treated with a nutritional composition comprising a mixture of 2′-FL and isomaltooligosacharide (IMO) in a 2:5 weight ratio, it remained unrevealed whether this increase was due to the administration of 2′-FL, IMO, or the combination.

The present invention therefore relates to the use of a preparation of dietary fibers for increasing the abundance of Faecalibacterium prausnitzii in the gastrointestinal tract of a human subject, the dietary fibers in said preparation consisting of 2′-fucosyllactose (2′-FL) and optionally one or more further oligosaccharides selected from the group consisting of galacto-oligosaccharides, fructo-oligosaccharides, polydextrose, resistant starch, and human milk oligosaccharides other than 2′-fucosyllactose. This use can be therapeutic or non-therapeutic.

The human subject is over 1 years of age, such as over 2, preferably over 3, more preferably over 18, particularly preferably over 40, even more preferably over 50, particularly more preferably over 60, and most preferably over 65 years of age.

The human subject preferably has a lower than average abundance of Faecalibacterium prausnitzii in the gastrointestinal tract.

The human subject may be a healthy human subject, but may also suffer from or have a high risk of developing inflammatory bowel conditions (such as inflammatory bowel diseases (IBD), ulcerative colitis or Crohn's disease), Covid-19 or a SARS-CoV-2 infection, diabetes, allergy (such as atopic dermatitis), anxiety, a sleep disorder, a lack of sleep, sleep deprivation, and/or an increased need for sleep.

A lower than average abundance of F. prausnitzii in the gastrointestinal tract as used herein refers to a lower number of F. prausnitzii in the gastrointestinal tract of a subject as compared to the average number of F. prausnitzii in the intestinal tract of a group of 10 healthy subjects of the same age group. A healthy subject is a subject that not diagnosed with a disease and is not suffering from any problems relating to the intestinal tract. Age groups are defined as from 0 up to 1 year old, from 1 up to 3 years old, form 3 up to 10 years old, from 10 up to 18 years old, from 18 up to 20 years old, from 20 up to 30 years old, from 30 up to 40 years old, from 40 up to 50 years old, from 50 up to 60 years old, from 60 up to 70 years old, from 70 up to 80 years old, form 80 up to 90 years old, and from 90 up to above 90 years old.

An increase in the abundance of F. prausnitzii in the gastrointestinal tract of a subject as used herein refers to a higher number of F. prausnitzii in the gastrointestinal tract of the subject after an intervention with 2′-FL (between 1 and 5 mg per day, for at least 2 weeks), as compared to prior to the intervention. The increase preferably is at least 3%, more preferably at least 8%, such as at least 10%, even more preferably at least 15%. The magnitude of the increase may depend on the daily dosage of 2′-FL. The abundance of F. prausnitzii may be determined as described below in the example.

The invention further relates to a preparation of dietary fibers consisting of 2′-fucosyllactose (2′-FL) and optionally one or more further oligosaccharides selected from the group consisting of galacto-oligosaccharides, fructo-oligosaccharides, polydextrose, resistant starch, and human milk oligosaccharides other than 2′-fucosyllactose, for use in increasing the abundance of Faecalibacterium prausnitzii in the gastrointestinal tract of a human subject suffering from or being at risk of developing COVID-19 or a SARS-CoV-2 infection, an inflammatory bowel condition such as inflammatory bowel diseases (IBD), ulcerative colitis, or Crohn's disease, diabetes, anxiety, and/or allergies such as atopic dermatitis.

The preparation is preferably administered to the human subject in a daily 2′-FL dosage of at least 0.01 grams, more preferably at least 0.02 grams, even more preferably at least 0.04 grams, more preferably at least 0.06 grams, more preferably at least 0.08 grams, even more preferably at least 0.10 grams, more preferably at least 0.5 grams, even more preferably at least 1.0 gram, and most preferably at least 2.0 grams.

The daily 2′-FL dosage is preferably at most 40 grams, more preferably at most 30 grams, even more preferably at most 20 grams, and most preferably at most 10 grams.

The preparation either contains solely of 2′-FL as dietary fiber or it contains, apart from 2′-FL, one or more oligosaccharides selected from the group consisting of galacto-oligosaccharides (GOS), fructo-oligosaccharides (FOS), resistant starch (RS), polydextrose (PDX) and human milk oligosaccharides other than 2′-fucosyllactose as dietary fibers.

Preferred oligosaccharides to be present in the preparation are GOS and/or FOS. The most preferred oligosaccharide is GOS.

The basic structure of galacto-oligosaccharide (GOS) includes a lactose core at the reducing end which is elongated with up to about seven galactose residues (degree of polymerization of 8; DP8).

Commercial GOS preparations are generally produced via a transgalactosylation reaction by enzymatic treatment of lactose with β-galactosidase enzymes (EC.3.2.1.23), yielding a mixture of oligomers with approximately 100 different types structures with varying DP and linkages. Beta-galactosidase is produced in many microorganisms such as Bacillus circulans, Aspergillus oryzae, Kluyveromyces marxianus, Kluyveromyces fragilis, Sporobolomyces singularis, Lactobacillus fermentum, and Papiliotrema terrestris (Cryptococcus Papiliotrema terrestris). Beta-galactosidases differ in their three-dimensional structures, resulting in stereo—and regioselective formation of the glycosidic bonds. After the enzymatic reaction, GOS is isolated and purified using conventional methods, such as nanofiltration and/or simulated moving bed chromatography (SMB). The resulting product is a GOS-containing syrup, which can be dried (e.g. by spray-drying, freeze-drying, or spray-cooling) to form a powder, if so desired.

Particularly preferred types of GOS are GOS prepared by a beta-galactosidase enzyme originating from Bacillus circulans, such as Biotis™ GOS. Beta-galactosidase originating from B. circulans possesses particularly strong transgalactosylation activity and is commercialized worldwide.

Fructo-oligosaccharides (FOS) are commercially produced by either inulin degradation or transfructosylation processes. Inulin is a naturally occurring polysaccharide produced by many types of plants, and is industrially most often extracted from chicory. Inulin is a polyfructose: a polymer of D-fructose residues linked by β(2→1) bonds with a terminal α(1→2) linked D-glucose. The degree of polymerization of inulin ranges from about 10 to about 60. Inulin can be enzymatically or chemically degraded to a mixture of oligosaccharides with the general structure Glu-Fruₙ (GFn) and Fruₘ (Fm), with n and m ranging from 1 to about 7. This process also occurs to some extent in nature, and these oligosaccharides can be found in a large number of plants, especially in Jerusalem artichoke, chicory, and the blue agave plant. The main components of commercial FOS are kestose (GF2), nystose (GF3), fructosylnystose (GF4), bifurcose (GF3), inulobiose (F2), inulotriose (F3), and inulotetraose (F4).

The second class of FOS is prepared by the transfructosylation action of a β-fructosidase (from, e.g. Aspergillus niger or Aspergillus) on sucrose. The resulting mixture has the general formula of GFₙ, with n ranging from 1 to 5.

Resistant starch (RS) is the fraction of dietary starch that escapes digestion in the small intestine.

Polydextrose (PDX) is a synthetically produced branched polymer of glucose units. Polydextrose is a form of soluble fiber and has shown healthful benefits.

Examples of HMOs other that 2′-FL include fucosylated lactoses such as 3′-fucosyllactose (3′-FL), sialylated lactoses such as 3′-sialyllactose (3′-SL) and 6′-sialyllactose (6′-SL), and tetrasaccharides like lacto-N-tetraose (LNT) and lacto-N-neotetraose (LNnT).

If the preparation of dietary fibers contains one or more oligosaccharides in addition to 2′-FL, it preferably comprises 2′-FL and the further oligosaccharides in a 2′-FL : further oligosaccharides weight ratio of 0.05:1-2:1, more preferably 0.08:1-2:1, even more preferably 0.10:1-1:1, even more preferably 0.20:1-0.50:1, and most preferably 0.25:1-0.5:1.

More preferably, this further oligosaccharide is FOS and/or GOS, meaning that the 2′-FL:(FOS+GOS) weight ratio is preferably 0.05:1-2:1, more preferably 0.08:1-2:1, even more preferably 0.10:1-1:1, even more preferably 0.20:1-0.50:1, and most preferably 0.25:1-0.50:1.

Most preferably, this further oligosaccharide is GOS and the 2′-FL:GOS weight ratio is preferably 0.05:1-2:1, more preferably 0.08:1-2:1, more preferably 0.10:1-1:1, even more, preferably 0.20:1-0.50:1, and most preferably 0.25:1-0.50:1.

The above weight-ratios are based on the dry weight of oligosaccharides (Dp≥2), thereby excluding monosaccharide contents.

It is theorized that when a mixture of dietary fibers, including GOS and 2′-FL, is administered to a human subject, a kind of cross-feeding occurs between the Bifidobacteria, in particular B. adolescensis and B. longum, and F. prausnitzii.

Furthermore, not only the F. prausnitzii abundance is decreasing during aging of human subjects, also the abundance of Bifidobacteria in the proximal colon decreases. The combined administration of 2′-FL and GOS would therefore further improve the microbiota composition and activity of human subjects.

In addition, there are strong indications that the abundance of F. prausnitzii may have an effect on the effectiveness on certain sleep treatments. It is known that dietary fibers, such as oligosaccharides, more in particular galacto-oligosaccharides (GOS), have an effect on sleep quality and/or sleep continuity. It has now been found that the gut microbiota in subjects positively responding to such treatment compared to those not responding to such treatment significantly differs. Although both responders and non-responders showed an increased abundance of Bifidobacteria—more specifically B. adolescensis and B. longum, which are known to produce metabolites that can impact the brain—the relative abundance of F. prausnitzii—which as such did not change during the treatment—in responders was about three times higher than in non-responders. Fecal samples of responders to said sleep treatment also showed a higher abundance of Feacalibacterium—related genetic pathways than those of non-responders. It is therefore expected that the response to treatment with dietary fibers is fortified by a baseline F. prausnitzii abundance above a certain level.

Therefore, it is expected that the increase in abundance of F. prausnitzii by the administration of a 2′-FL preparation will increase the responsiveness of human subjects—i.e. increase the number of responders—to the positive effect of dietary fibers, such as oligosaccharides, more in particular galacto-oligosaccharides (GOS), on sleep quality and/or sleep continuity.

In order to achieve this effect, 2′-FL may be administered together with said dietary fibers; in a combined preparation or nutritional composition or in separate preparation or nutritional compositions administered during the same treatment period. Alternatively, 2′-FL administration may be started days or weeks prior to the administration of said dietary fibers in order to increase the baseline levels of F. prausnitzii, thereby increasing the responsiveness to the effect of the dietary fibers on sleep quality and/or continuity.

“Improving sleep quality and/or sleep continuity” includes one or more, preferably two or more of the following aspects: promoting falling asleep; inducing or supporting a mature sleep pattern; reducing or preventing sleep disturbances/sleeping more time while in bed; increasing the feeling of a deep sleep; feeling more refreshed at waking up; and/or feeling more energized and/or having a better mood during daytime.

The present invention therefore also relates to the (non-)therapeutic use of the 2′-FL-containing preparation for improving sleep quality and/or sleep continuity. It also relates to the (non-)therapeutic use of the 2′-FL-containing preparation for enhancing the effect of dietary fibers, preferably oligosaccharides, more preferably galacto-oligosaccharides (GOS), on sleep quality and/or sleep continuity.

The preparation of dietary fibers can be administered as such, but is preferably administered as part of a nutritional composition. In such nutritional composition, the dietary fibers are preferably present in an amount of at least 10 wt. %, e.g. at least 20 wt. %, at least 30 wt. %, at least 40 wt. %, at least 50 wt. %, at least 60 wt. %, at least 70 wt. %, at least 80 wt. %, or at least 90 wt. %, e.g. up to 91 wt %, 92 wt %, 93 wt %, or 94 wt %, relative to the total weight of the nutritional composition and based on dry weight. More preferably, 2′-FL is preferably present in the nutritional composition in an amount of at least 10 wt. %, e.g. at least 20 wt. %, at least 30 wt. %, at least 40 wt. %, at least 50 wt. %, at least 60 wt. %, at least 70 wt. %, at least 80 wt. %, or at least 90 wt. %, e.g. up to 91 wt %, 92 wt %, 93 wt %, or 94 wt %, relative to the total weight of the nutritional composition and based on dry weight.

The nutritional composition can have the form of a food product, a beverage, or a dietary supplement.

The nutrition composition comprises, apart from the preparation of dietary fibers, at least one further ingredient selected from lipids, digestible carbohydrates, probiotics, proteins, and/or additional nutritional agents, such as vitamins, minerals, and/or biologically active peptides.

Apart from the preparation of dietary fibers, the nutritional composition does not contain any material amounts of additional dietary fibers. That is, not more than 5 wt %, more preferably not more than 1 wt % of the total amount of dietary fibers in the nutritional composition may result from sources other than the preparation of dietary fibers.

Examples of lipids are animal lipids (milk fat, fish oil) and/or vegetable lipids (e.g. algae oil, fungal oil, and bacterial oil) and preferably include long chain poly-unsaturated fatty acids (LC-PUFA; fatty acids or fatty acyl chains with a length of 20 to 24 carbon atoms, preferably 20 or 22 carbon atoms comprising two or more unsaturated bonds).

Examples of proteins are milk proteins (e.g. casein and/or whey protein), plant proteins (e.g. soy protein and/or rice protein), hydrolysates thereof such as polypeptides with up to about 20 amino acids in length (like casein tryptic hydrolysates), fermented products thereof (such as fermented whey protein concentrate or fermented whey protein isolate), free amino acids such as tryptophan and cysteine, and mixtures thereof.

Examples of digestible carbohydrates are sucrose, lactose, glucose, fructose, corn syrup solids, starch, and maltodextrins.

Examples of vitamins and minerals are magnesium, zinc, vitamin B3 and vitamin B6, and, in view of the oxygen sensitivity of F. prausnitzii, vitamins providing anti-oxidant effects, such as vitamin C, vitamin E, and/or beta carotene.

The nutritional composition may further contain flavoring agents, preservatives and/or colouring agents.

Examples of suitable probiotics are (synbiotic) bacteria such as Bifidobacteria and/or Lactobacilli.

The nutritional composition may have a liquid, semi-liquid, or solid constituency.

The nutritional composition is not milk of a mammal, such as milk from a human, goat, sheep, cow, camel, or ruminant. The nutritional composition is a synthetic composition. The term “synthetic composition” designates a composition which is artificially prepared and preferably means a composition containing at least one compound that is produced ex vivo chemically and/or biologically, e.g. by means of chemical reaction, enzymatic reaction or recombinantly.

Examples of suitable food products and beverages are dairy products, such as milk, milkshake, chocolate milk, yoghurt, cream, cheese, pudding, and ice cream; bars, such as nutritional bars, energy bars, snack bars, cereal bars, and bars for diabetics; liquid products, such as nutritional drinks, diet drinks, liquid meal replacers, sports drinks, and other fortified beverages; savory snacks, such as chips, tortillas, puffed and baked snacks, crackers, pretzels, and savory biscuits; bakery products, such as muffins, cakes, and biscuits; sweets such as gummies; and pastas, such as spaghetti.

Food supplements can have the form of pills, capsules, or dry powders. Food supplements may be ready for consumption or may need to be dissolved in a liquid like water. The product in dry powder form may be accompanied with a device, such as a spoon, to measure the desired amount of the powder (e.g. daily or unit dose). The nutritional composition may be provided in a jar, bottle, sachet, carton, rapping, and the like.

In a preferred embodiment, the preparation of dietary fibers or the nutritional composition comprising said preparation are provided in the form of a single serving. Each single serving may optionally be individually packaged.

In one embodiment, a single serving preferably comprises 1.0-40 grams, preferably 1.0-30 grams, preferably 1.5-25 grams, more preferably 2.0-20 grams, more preferably 2.5-15 grams, even more preferably 3.0-10 grams, more preferably 3.5-8 grams, and most preferably 4.0-5.0 grams 2′-FL.

In another embodiment, a single serving preferably comprises at least 0.01 grams, more preferably at least 0.02 grams, even more preferably at least 0.04 grams, more preferably at least 0.06 grams, more preferably at least 0.08 grams, even more preferably at least 0.10 grams, more preferably at least 0.5 grams, even more preferably at least 1.0 gram, and most preferably at least 2.0 grams 2′-FL. The single serving according to this embodiment preferably comprises at most 40 grams, more preferably at most 30 grams, even more preferably at most 20 grams, and most preferably at most 10 grams 2′-FL.

Unit doses of the preparation or nutritional composition are preferably administered at least once a week, preferably at least once every 3 days, more preferably at least once every other day, most preferably at least once daily.

In one embodiment, the daily dosage of 2′-FL is preferably in the range 0.5-40 grams, preferably 0.5-30 grams, more preferably 0.5-20 grams, more preferably 0.5-10 grams, even more preferably 1.0-10 grams, more preferably 1.0-5.0 grams, and most preferably 4.0-5.0 grams 2′-FL.

In another embodiment, the daily dosage of 2′-FL is preferably in at least 0.01 grams, more preferably at least 0.02 grams, even more preferably at least 0.04 grams, more preferably at least 0.06 grams, more preferably at least 0.08 grams, and most preferably at least 0.10 grams 2′-FL. The daily dosage according to this embodiment is preferably at most 10 grams, more preferably at most 8.0 grams, even more preferably at most 6.0 grams, more preferably at most 5.0 grams, more preferably at most 4.0 grams, more preferably at most 2.0 grams, and most preferably at most 1.0 gram 2′-FL.

The use of the preparation or nutritional composition is preferably continued for a period of at least two weeks, e.g. at least 3 weeks, at least 4 weeks, at least 1 month, at least two months, at least three months, at least 4 months, at least 5 months, or even at least 6 months. In order to maintain the health benefits caused by the administration of the preparation or nutritional composition, the preparation or nutritional composition is preferably administered at least once per day. It may be taken together with a meal such as during breakfast or at the end of the day, e.g. 0-120 minutes, more preferably 0-60 minutes, and most preferably 0-30 minutes before going to bed. Alternatively, it may be administered twice per day, preferably in the morning and in the evening, e.g. during breakfast and dinner or during breakfast and before going to bed. Administration together with a meal is convenient, and lowers the risk that consumers forget to take the preparation or nutritional composition.

### EXAMPLES

### Example 1

A group of twelve randomly contacted adults—three men and three women aged 25-50 yrs and three men and three women aged 70-79 yrs—donated their fecal material for an in vitro study. Anoxically cryo-conserved fecal inoculum was defrosted and transferred to a 96% N₂ and 4% H₂ filled anaerobic chamber. Incubation of 2′-FL (10 mg/ml) was done with 10% (v/v) fecal inoculum in duplicate, while incubations without fecal inoculum and without carbohydrates served as controls using the standard ileal efflux medium. Samples were collected 0 h, 4 h, 10 h and 24 h after inoculation.

Three aliquots of 1 ml from each culture bottle were then distributed into 1.5 ml Eppendorf tubes. One of these aliquots was heated at 100° C. for 5 min to determine carbohydrates in the supernatant. Afterwards, all samples were centrifuged at 4° C. at 18600 relative centrifugal force (rcf) for 10 min. The supernatants from the other two tubes were pooled and stored at −20° C. for metabolite measurement, while the remaining pellets were stored at −80 C for microbiota composition analysis.

The microbiota composition was determined by sequencing of barcoded 16S ribosomal RNA (rRNA) gene amplicons using Illumina Hiseq2500 (2×150 bp). Collected pellets were mixed with 350 μl Stool Transport and Recovery (STAR) buffer (Roche Diagnostics, United States) and subsequently transferred into a screw cap tube containing 0.25 g of 0.1 mm zirconia beads and 3 glass beads (diameter 2.5 mm).

Samples were then subjected to repeated bead beating (3 times 5.5 ms×60 s) using the FastPrep-24™ 5G bead beating grinder and lysis system (MP Biomedicals, the Netherlands) and followed by 15 min centrifugation at 4° C. Supernatant was collected, and the pellet was subjected to another cycle of isolation with 300 μl STAR buffer. 250 μl of combined supernatants was transferred into the MaxwellR 16Tissue LEV Total RNA purification Kit Cartridge (XAS1220) and processed using the MaxwellR 16 Instrument (Promega, Leiden, The Netherlands).

Subsequently, DNA was eluted in 35 μl of nuclease free water. The V4 region of the 16S rRNA gene was amplified in triplicate using barcoded 515F -806R primers and diluted DNA (in nuclease free water, 20 ng/μl) as template with an annealing temperature of 50° C. The PCR was performed as described by R. An, et al., Nutrients, 11 (2019) 2193. An equimolar mix of purified PCR products was sent for sequencing (Eurofins Genomics, Konstanz, Germany). Raw sequencing data was processed using NG-Tax 1.0. Taxonomy was assigned based on SILVA database version 128.

To compare and contrast alterations in microbiota composition with different carbohydrates versus no-carbohydrate control during the incubation, principal response curve (PRC) analysis was used to identify genera which fit the best (weights>0.05) to explain the observed difference between no-carbohydrate and carbohydrate-based incubation, using the prc function in the vegan package [J. Oksanen, et al., Package ‘vegan’. Community ecology package, version, 2013, 2(9)].

This resulted in the observation that the intake of 2′-FL leads to a higher abundance of F. prausnitzii as compared to the outgrowth on the control medium without 2′-FL.

### Example 2

This study was designed as a double-blind randomized placebo-controlled cross-over study in 70 apparently healthy Dutch adults (age 30-50 y, BMI 19.5-25 kg/m²) with sleep problems (Pittsburg Sleep Quality Index (PSQI) ≥9).

Included subjects were randomized (all at the same time) to start with either Biotis™ Sleepwell or a placebo, each for 3 weeks with a washout period of 3 weeks.

Biotis™ Sleepwell is a composition comprising 19.3 wt % Biotis™ GOS, 10 wt % lactose, and 52.5 wt % whey protein.

The placebo was skimmed milk powder.

Biotis™ Sleepwell and the placebo were packaged in identical white sachets containing 16.3 grams of product. In order to mask taste differences, the products were vanilla flavoured and sweetened with sucralose. The products had to be dissolved in 150 ml lukewarm water, and were consumed once daily, about 1 hour before going to bed.

During each intervention period, a daily questionnaire on bedtime and wake-up time, lifestyle habits, sleep characteristics (PSQI questionnaire), and fitness and mood at waking up had to be filled in. Sleep tracker (SmartSleep™, Philips, The Netherlands) measurements took place during 5 consecutive nights, before product consumption, during each intervention period, and during the last 5 nights of each period. Stool samples were collected during the 1^{st} intervention period only, at days 0 and 21.

DNA as isolated from the 138 fecal samples of the intervention study were subjected to full (5 Gb) shotgun metagenomic sequencing (PE150 reads, Novaseq 6000). Raw sequencing data were processed for shotgun metagenomic sequencing data (HUMANn2 workflow-based), including quality control (QC). Raw sequencing data were analyzed in a bioinformatics workflow for shotgun metagenomic sequencing, which includes data quality control, mining, and representation of the output by means of Visual Analytics including biological interpretation of the data.

Microbiome composition was analyzed using multivariate (PCA/RDA) and bivariate statistical methods. Comparisons were made between treatment groups, and between subjects that slept relatively well and that slept relatively bad, before and after intervention with the intervention product. Microbiome genetic functions (detected by the generic bioinformatics pipeline and background database) in the samples were exploratively compared in the same way.

A targeted analysis on microbial functions that have been linked with sleep quality in literature was performed with dedicated bioinformatics modules. For this purpose, the sequencing reads were assembled into contigs (per-sample de novo assembly). The following modules were used:
1) the “Gut-Brain” module as published by M. Valles-Colomer, et. al., Nature Microbiology 4 (2019) 623-632, was used to search for this type of genes and to quantify (relative abundance) them.
2) The “Gut-Brain” module was extended by newly build bioinformatics modules specifically looking for microbial genes that have been associated with sleep quality, more specifically genes involved in GABA production, deconjugation of catecholamines and histidine decarboxylation. The gene list was used for the generation of novel functional bioinformatics modules based on Hidden Markov Models (HMMs). Putative genes were identified in the microbiome data by scanning predicted protein sequences with the HMMs. Downstream statistical analysis was performed the same way as described for compositional analysis.

### Results

Within the study group, there appeared to be true responders—i.e. subjects observing sleep improvement after taking Biotis™ Sleepwell, but not when taking the placebo—and true non-responders—i.e. subjects showing neither sleep improvement with Biotis™ Sleepwell, nor with the placebo.

The microbiota of these two groups were compared and significant taxonomic compositional differences were revealed.

Before the start of the intervention (day 0), the median relative abundance of Bifidobacterium longum was approximately 3 times lower in true responders (2.5%) as compared to true non-responders (7.5%) and the median relative abundance of Bifidobacterium adolescentis was approximately 7 times lower in true responders (1.2%) as compared to true non-responders (8.6%).

After three weeks of intervention with Biotis™ Sleepwell, a significant increase in beneficial Bifidobacterium was detected in both true responders and true non-responders. This evidences the effect of Biotis™ Sleepwell, more in particular GOS, on Bifidobacteria abundance.

However, the median relative abundance of Bifidobacterium genus levels in true responders remained significantly lower (12.7%) than in true non-responders (25.8%). The same trend was found for the Bifidobacteriaceace family. In other words: the Bifidobacteria abundance as such does not directly correlate with sleep.

It was also observed that the median relative abundance of Faecalibacterium prausnitzii at day 0 was approximately 3 times higher in true responders (18.2%) as compared to true non-Responders (6.2%).

No stimulating effect of the Biotis™ Sleepwell on Faecalibacterium prausnitzii was detected at the end of the study; neither in true reponders, nor in true non-reponders.

The microbial pathways (MetaCyc) of true responders and true non-responders were also compared. At day 0, 26 MetaCyc pathways were different between true responders and true non-responders, these pathways having a strong correlation with Faecalibacterium prausnitzii: the Curated Gut-Brain Modules (GBMs; as described by M. Valles-Colomer, et. al., Nature Microbiology 4 (2019) 623-632,), “quinolinic acid degradation” (increased quinolic acid is linked to depression; O. V. Averina et al., Int. J. Mol. Sci., 21 (2020) 9234), “inositol synthesis” (frontal cortex inositol positively correlated to total sleep time and negatively with depression severity; A. S. Urrila et al., Neurospychobiology 75 (2017) 21-31) and “nitric oxide synthesis II—nitrite reductase” (Nitric oxide is considered a beneficial neurotransmitter) were significantly lower in true responders.

The above strongly suggests that (i) treatment with GOS increases the Bifidobacteria abundance, but (ii) this only results in improved sleep if the baseline Faecalibacterium prausnitzii adundance is at a certain minimum level. Hence, increasing this baseline level, which can be done with 2′-FL (see Example 1), is expected to increase the number of true responders to a treatment with GOS.

Examples of such compositions are presented in the following tables:

| Nutrient | content |
|---|---|
| Protein (g) | 10 |
| whey protein (g) | 9.5 |
| Lactium ® (73% | 200 |
| protein) (mg) | |
| Lactose (g) | 1.9 |
| GOS (g) | 5.2 |
| 2′FL (g) | 0.5 |
| Magnesium (mg) | 200 |
| Zinc (mg) | 5 |
| Vitamin B6 (mg) | 1 |
| Niacin (mg) | 10 |
| Vitamin D3 (μg) | 10 |

| Nutrient | content |
|---|---|
| Protein (g) | 10 |
| whey protein (g) | 9.5 |
| Lactium ® (73% | 200 |
| protein) (mg) | |
| Lactose (g) | 1.9 |
| GOS (g) | 5.2 |
| 2′FL (g) | 0.25 |
| Magnesium (mg) | 200 |
| Zinc (mg) | 5 |
| Vitamin B6 (mg) | 1 |
| Niacin (mg) | 10 |
| Vitamin D3 (μg) | 10 |

| Nutrient | content |
|---|---|
| Protein (g) | 10 |
| whey protein (g) | 9.5 |
| Lactium ® (73% | 200 |
| protein) (mg) | |
| Lactose (g) | 1.9 |
| GOS (g) | 5.2 |
| 2′FL (g) | 1.0 |
| Magnesium (mg) | 200 |
| Zinc (mg) | 5 |
| Vitamin B6 (mg) | 1 |
| Niacin (mg) | 10 |
| Vitamin D3 (μg) | 10 |

1. A method of increasing the abundance of Faecalibacterium prausnitzii in the gastrointestinal tract of a human subject, the method comprising administering to the human subject an effective amount of a preparation of dietary fibers, the dietary fibers in said preparation consisting essentially of 2′-fucosyllactose (2′-FL) and optionally one or more further oligosaccharides selected from the group consisting of galacto-oligosaccharides, fructo-oligosaccharides, resistant starch, polydextrose, and human milk oligosaccharides other than 2′-fucosyllactose.

2. The method of claim 1, wherein the human subject is over 1 years of age.

3. The method of claim 1, wherein the human subject has a lower than average abundance of Faecalibacterium prausnitzii in the gastrointestinal tract prior to administration of the preparation.

4. The method of claim 1, wherein administering the preparation to the human subject has an effect of improving sleep quality and/or sleep continuity in the human subject.

5. The method of claim 1, wherein administering the preparation to the human subject has an effect of enhancing the effect of dietary fibers on sleep quality and/or sleep continuity in the human subject.

6. The method of claim 1, wherein the preparation is administered to said human subject in a daily 2′-FL dosage of at least 0.01 grams.

7. The method of claim 1, wherein the preparation comprises 2′-fucosyllactose (2′-FL) and galacto-oligosaccharides (GOS) in a 2′-FL:GOS weight ratio of 0.05:1-2:1.

8. The method of claim 1, wherein the preparation is part of a nutritional composition additionally comprising lipids, digestible carbohydrates, proteins, and/or vitamins.

9. A method of increasing the abundance of Faecalibacterium prausnitzii in the gastrointestinal tract of a human subject suffering from or being at risk of developing COVID-19 or a SARS-CoV-2 infection, an inflammatory bowel condition, diabetes, anxiety, and/or allergies, the method comprising administering to the human subject an effective amount of a preparation of dietary fibers consisting essentially of 2′-fucosyllactose (2′-FL) and optionally one or more further oligosaccharides selected from the group consisting of galacto-oligosaccharides, fructo-oligosaccharides, resistant starch, and human milk oligosaccharides other than 2′-fucosyllactose.

10. The method of claim 9, wherein the preparation comprises 2′ fucosyllactose (2′-FL) and galacto-oligosaccharides (GOS) in a 2′-FL:GOS weight ratio of 0.05:1-2:1.

11. The method of claim 9, wherein the preparation is administered to said human subject in a daily 2′-FL dosage of at least 0.01 grams.

12. The method of claim 9, wherein the preparation is administered to said human subject in a daily 2′-FL dosage of at most 40 grams.

13. The method of claim 9, wherein the preparation is part of a nutritional composition additionally comprising lipids, digestible carbohydrates, proteins, probiotics, and/or vitamins.

14. The method of claim 6, wherein the preparation is administered to said human subject in a daily 2′-FL dosage of at least 0.10 grams.

15. The method of claim 7, wherein the preparation comprises 2′-fucosyllactose (2′-FL) and galacto-oligosaccharides (GOS) in a 2′-FL:GOS weight ratio of 0.25:1-0.5:1.

16. The method of claim 9, wherein the inflammatory bowel condition comprises inflammatory bowel diseases (IBD), ulcerative colitis, or Crohn's disease.

17. The method of claim 9, wherein the allergies comprise atopic dermatitis.

18. The method of claim 10, wherein the preparation comprises 2′-fucosyllactose (2′-FL) and galacto-oligosaccharides (GOS) in a 2′-FL:GOS weight ratio of 0.25:1-0.5:1.

19. The method of claim 11, wherein the preparation is administered to said human subject in a daily 2′-FL dosage of at least 2.0 grams

20. The method of claim 12, wherein the preparation is administered to said human subject in a daily 2′-FL dosage of at most 10 grams.
